(19) United States
(12) Patent Application Publication
(10) Pub. No.: US 2023/0346619 A1
(43) Pub. Date: Nov. 2, 2023

(54) Lifting/Lowering Underframe and Load-bearing Device Having Same

(71) Applicant: DONGPIN SALON & BEAUTY EQUIPMENT CO., LTD, FoshanCity, Guangdong Province (CN)

(72) Inventors: Guo Liang Li, Foshan City (CN); Guo Ling Li, Foshan City (CN); Yao Cong Pan, Foshan City (CN)

(21) Appl. No.: 17/921,327

(22) PCT Filed: Apr. 28, 2021
(86) PCT No.: PCT/CN2021/090481

§ 371 Date: Oct. 25, 2022

(30) Foreign Application Priority Data
Apr. 29, 2020 (CN) 202010356662.8

(51) Int. Cl. A61G 13/06; A61G 13/04

(52) U.S. Cl. CPC A61G 13/06; A61G 13/04

(57) ABSTRACT

A lifting/lowering underframe and a load-bearing device having same. The lifting/lowering underframe comprises a lifting/lowering mechanism (2) which comprises a first lifting/lowering frame (21), a second lifting/lowering frame (22) and a driving member (23). The two ends of the driving member (23) are connected to the first lifting/lowering frame (21) and the second lifting/lowering frame (22) respectively; the driving member (23) is an electric push rod, and the driving member (23) kept between the first lifting/lowering frame (21) and the second lifting/lowering frame (22) can electrically drive the first lifting/lowering frame (21) and the second lifting/lowering frame (22) to move relative to each other. The lifting/lowering mechanism (2) is easy to maintain, has low noise and can work normally in high/low temperature and anticorrosion/explosion-proof harsh environments.

### BACKGROUND OF THE PRESENT INVENTION

### Field of Invention

The invention relates to the field of mechanical equipment, in particular to a lifting/lowering underframe and a load-bearing device having same.

### Description of Related Arts

With the development of the times, super mainstream fashion such as beauty salons and tattoos has also entered the life of ordinary people, especially tattoos, etc., which have also developed from the symbol of street gangsters to the current mainstream of super fashion, and are deeply sought after and loved by young friends. Since the position of the tattoo can be on the outer surface of any part of the body, the tattooing bed is generally used as the basic equipment in order to perform tattoo operations on various parts of the body.

At present, the shape and function of the bed used for tattooing are single, as shown in Chinese Patent No. 201620855490.8, which discloses a tattooing bed that can diversely adjust. The tattooing bed comprises a back portion bed board, a middle part bed board, a shank bed board and a bed bottom plate. A lifting and lowering device composed of gas pole is arranged between the back portion bed board and the middle part bed board and between the middle part bed board and the shank bed board, and an oil pressure pump elevating gear who sets up between bed board and the bed bottom plate.

In order to realize the height lifting and lowering of the tattooing bed, the above-mentioned tattooing bed includes a lifting and lowering device provided thereof. Most of the tattooing beds or tattooing chairs on the market today are driven by air cylinders or oil cylinders. However, the driving and lifting of the air cylinder or oil cylinder requires a complex complete set of system support, especially the oil cylinder drive, which requires a combination of components such as pumps, pipes, valves, filters, joints, etc., and it takes up a lot of space, the maintenance work is cumbersome, the cost is high, and the operation process is noisy.

### SUMMARY OF THE PRESENT INVENTION

An object of the present invention is to provide a lifting/lowering mechanism with a simple structure and good performance, and a lifting/lowering underframe using the same, which can solve at least one of the above problems.

Another object of the present invention is to provide a lifting/lowering underframe and a load-bearing device having same, wherein the lifting/lowering underframe can be raised and lowered by electric drive, the structure is simple and lightweight, and the maintenance is easy.

Another object of the present invention is to provide a lifting/lowering underframe and a load-bearing device having same, wherein the lifting/lowering underframe can be switched between a rotatable state and a locking state, wherein a lifting/lowering mechanism of the lifting/lowering underframe in the rotatable state can rotate relative to an affixed base, thereby changing the orientation of a load- bearing device installed on the lifting/lowering mechanism; the lifting/lowering mechanism of the lifting/lowering underframe in the locked state cannot be rotated, so that the orientation of the load-bearing device is affixed.

Another object of the present invention is to provide a lifting/lowering underframe and a load-bearing device having same, wherein the lifting/lowering mechanism can rise and fall smoothly and has better stability.

Another object of the present invention is to provide a lifting/lowering underframe and a load-bearing device having same, wherein the lifting/lowering underframe is allowed to be manually operated to switch between the rotatable state and the locking state.

Another object of the present invention is to provide a lifting/lowering underframe and a load-bearing device having same, wherein the lifting/lowering underframe is allowed to electrically switch between the rotatable state and the locking state.

According to an aspect of the present invention, the present invention provides a lifting/lowering mechanism, comprising a first lifting/lowering frame, a second lifting/lowering frame and a driving member, wherein the first lifting/lowering frame is sleeved and installed on the second lifting/lowering frame, wherein the driving member is an electric push rod, and is installed on one of the first lifting/lowering frame or the second lifting/lowering frame, so as to drive the first lifting/lowering frame and the second lifting/lowering frame to move relative to each other.

Therefore, the lifting/lowering mechanism of the present invention is driven by the electric push rod, which can save the use space without the support of a complex complete system, and the lifting/lowering mechanism can work safely and reliably without protection. The lifting/lowering mechanism of the present invention is simple to maintain, has low noise, and can work normally in high/low temperature, and anti-corrosion/explosion-proof harsh environments.

According to some embodiments, the first lifting/lowering frame includes an upper mounting plate and a first lifting/lowering pipe mounted on the upper mounting plate, and the first lifting/lowering pipe is at least one, wherein the second lifting/lowering frame includes a lower mounting plate and a second lifting/lowering pipe mounted on the lower mounting plate, and the second lifting/lowering pipe is at least one, wherein the second lifting/lowering pipe is sleeved on the first lifting/lowering pipe, and the two ends of the driving member are respectively connected with the upper mounting plate and the lower mounting plate. Thereby, the smooth progress of the lifting/lowering process can be ensured, and the safety is further improved.

According to some embodiments, the 1 lifting/lowering mechanism further includes a rotation adjusting component and a bottom plate, wherein the rotation adjusting component includes a limiting block, wherein the lower mounting plate is a gear plate, wherein the limiting block and the lower mounting plate are both rotatably installed on the bottom plate, the limiting block is located on the outer side of the lower mounting plate, and the limiting block cooperates with the lower mounting plate in position limit. Consequently, the lifting/lowering mechanism of the present invention not only has a height lifting/lowering function, but also has a rotation adjustment function and a rotation limit function, which has stronger applicability and more complete functions, and can meet various usage requirements.

According to some embodiments, the limiting block has teeth arranged thereon that cooperate with the lower mounting plate. Therefore, the limiting block allows better limit locking of the the lower mounting plate.

According to some embodiments, the lower mounting plate is rotatably mounted on the bottom plate through a rotating shaft. Therefore, the lower mounting plate is allowed to drive the entire lifting/lowering mechanism to rotate, so as to meet the usage requirements.

According to some embodiments, a gasket is provided between the lower mounting plate and the bottom plate. Hence, the gasket is capable of effectively avoiding the direct contact between the lower mounting plate and the bottom plate, thereby prolonging the service life of each. The gasket of the present invention can be a nylon gasket, and the nylon gasket is low cost and durable.

According to another aspect of the present invention, the present invention provides a lifting/lowering underframe, which includes the above-mentioned lifting/lowering mechanism, and further includes a chassis, a first casing and a second casing, wherein the second lifting/lowering frame is installed on the chassis through the bottom plate, wherein the first casing is sleeved on the outer periphery of the first lifting/lowering frame, wherein the second casing is sleeved on the outer periphery of the second lifting/lowering frame, and the first casing is sleeved on the second casing.

Therefore, the lifting/lowering underframe of the present invention adopts the above-mentioned lifting/lowering mechanism, when the lifting/lowering underframe needs to be lifted and lowered, the driving member drives the first lifting/lowering frame to move relative to the second lifting/lowering frame, so as to drive the first casing to move relative to the second casing, thereby realizing the height adjustment of the first casing and meeting the requirements of use. The lifting/lowering underframe of the present invention integrates lifting, rotating and rotating locking functions, has simple and compact structure, stable running process and convenient operation, and is very suitable for application in fields such as tattooing beds or tattooing chairs.

According to some embodiments, the rotation adjusting component further includes an adjustment handle, a linkage rod and an affixed rod, wherein the affixed rod is fixedly installed on the chassis, wherein one end of the linkage rod is matched with the limiting block, and the other end of the linkage rod is connected with the adjustment handle. The linkage rod is hinged with the affixed rod. Thus, the linkage rod is driven to rotate by the adjustment handle, thereby driving the limiting block to rotate, so that the teeth on the limiting block are away from or fastened to the lower mounting plate, so as to realize the unlocking and locking functions. The invention adopts the linkage mode to realize the limit adjustment, so that the operation is convenient.

According to some embodiments, the limiting block has a first limiting groove arranged thereon, and the linkage rod has a limiting column matched with the first limiting groove arranged at one end thereof. Accordingly, the limiting column is capable of sliding in the first limiting groove, thereby pushing the limiting block to move to complete the corresponding action.

According to some embodiments, the chassis has a second limiting groove arranged thereon, wherein one end of the adjustment handle is fixedly connected with the linkage rod, and the other end passes through the second limiting groove and is capable of moving in the second limiting groove. Therefore, the second limiting groove is capable of limiting the adjustment handle.

According to an embodiment of the present invention, the present invention provides a lifting/lowering underframe, which comprises:
a lifting/lowering mechanism, which comprises a first lifting/lowering frame, a second lifting/lowering frame and a driving member, wherein two ends of the driving member are respectively connected with the first lifting/lowering frame and the second lifting/lowering frame, wherein the driving member is an electric push rod, wherein the driving member held between the first lifting/lowering frame and the second lifting/lowering frame allows the relative movement of the first lifting/lowering frame and the second lifting/lowering frame to be driven electrically.

According to an embodiment of the present invention, the first lifting/lowering frame comprises an upper mounting plate and at least one first lifting/lowering pipe extending downward from the upper mounting plate, wherein the second lifting/lowering frame comprises a lower mounting plate and at least one second lifting/lowering pipe mounted on the lower mounting plate, wherein the first lifting/lowering frame is held above the lower mounting plate in a manner that the first lifting/lowering pipe is movably mounted on the second lifting/lowering pipe, wherein two ends of the driving member are respectively connected with the upper mounting plate and the lower mounting plate.

According to an embodiment of the present invention, the lifting/lowering underframe further comprises a limiting block and a bottom plate, wherein the lower mounting plate and the limiting block are rotatably mounted on the bottom plate, and the limiting block is located an outer side of the lower mounting plate, so that the limiting block is allowed to limit the rotation of the lower mounting plate relative to the bottom plate.

According to an embodiment of the present invention, the limiting block of the lifting/lowering underframe has a plurality of mutually spaced tooth slots formed inwardly recessed on the surface thereof, wherein the limiting block has at least one tooth matched with the tooth slots and arranged thereon.

According to an embodiment of the present invention, the lifting/lowering underframe comprises a chassis for fixing the bottom plate, a first casing sleeved and mounted on the first lifting/lowering frame, and a second casing sleeved and mounted on the second lifting/lowering frame, wherein the first casing is movably mounted to the second casing.

According to an embodiment of the present invention, the lifting/lowering underframe further comprises a affixed rod fixed to the chassis, a linkage rod movably arranged on the affixed rod, and an adjustment handle arranged at one end of the linkage rod, wherein the other end of the linkage rod is connected with the limiting block, so that the linkage rod is capable of driving the limiting block to move relative to the lower mounting plate.

According to an embodiment of the present invention, the lifting/lowering underframe further comprises a limiting column arranged at the end of the linkage rod, wherein the limiting block has a first limiting groove arranged thereon, wherein the limiting column is movably held in the first limiting groove of the limiting block.

According to an embodiment of the present invention, the chassis has a second limiting groove arranged thereon, wherein the adjustment handle is movably held in the second limiting groove.

According to another aspect of the present invention, the present invention provides a lifting/lowering underframe, which comprises:
a lifting/lowering mechanism, which comprises a rotating member, a maintaining member and at least one driving member, wherein the two ends of the driving member are respectively connected with the rotating member and the maintaining member, so that the driving member is capable of driving the rotating member and the maintaining member to move relative to each other; and
a affixed base, wherein the lifting/lowering mechanism is rotatably mounted on the affixed base, and allows the lifting/lowering underframe to be capable of switching between a rotatable state and a locking state.

According to an embodiment of the present invention, the lifting/lowering mechanism further comprises at least one holding member, wherein the holding member comprises a first holder affixed to the rotating member, and a second holder affixed to the maintaining member, wherein the second holder is movably mounted to the first holder.

According to an embodiment of the present invention, the lifting/lowering underframe further comprises a linear bearing mounted on the first holder or the second holder.

According to an embodiment of the present invention, the driving member is implemented as two, wherein the two driving members are symmetrically arranged on the rotating member and the maintaining member.

According to an embodiment of the present invention, the lifting/lowering underframe further comprises a limiting member, wherein the rotating member comprises an assembly portion, a plurality of mutually spaced limiting teeth extending outward from the edge of the assembly portion, and a limiting tooth slot is formed between the two adjacent limiting teeth and the assembly portion, wherein the limiting member is movably held on the outer side of the rotating portion, and at least a part of the limiting member is allowed to enter the limiting tooth slot.

According to an embodiment of the present invention, the limiting member comprises a rotating portion, a limiting arm bent outwardly extending from the rotating portion, and at least one limiting protrusion extending outwardly from an inner wall of the limiting arm, so that after the limiting member is driven close to the rotating member, the limiting protrusion of the limiting arm is allowed to enter the limiting tooth slot of the rotating member to limit the rotation of the rotating member.

According to an embodiment of the present invention, the lifting/lowering underframe further comprises an operating member, wherein the limiting member is drivably connected with the operating member, so that the operating member is capable of driving the limiting member to approach the rotating member and move away from the rotating member.

According to an embodiment of the present invention, the limiting member has a driving space formed in the limiting arm, wherein the operating member comprises a connecting arm, a limiting column and an operating portion, wherein the limiting column and the connecting arm respectively extend outward from two ends of the connecting arm, wherein the connecting arm is rotatably mounted on the affixed base in a manner that the limiting column is movably held in the driving space of the limiting member, wherein the connecting arm rotates relative to the affixed base, which is capable of driving the limiting column to move in the driving space of the limiting member, and drive the limiting member to be close to the rotating member or away from the rotating member.

According to an embodiment of the present invention, the driving space has a driving proximal end and a driving distal end relative to the driving proximal end, wherein the vertical distance between the driving proximal end and the limiting protrusion is greater than the vertical distance between the driving distal end and the limiting protrusion.

According to an embodiment of the present invention, the driving space is an arc-shaped groove.

According to an embodiment of the present invention, the operating member comprises a control member and an electric telescopic member, wherein the control member is communicatively connected with the electric telescopic member, wherein the limiting member is arranged at an end of the electric telescopic member, wherein the operating member electrically drives the limiting member to move relative to the rotating member.

According to an embodiment of the present invention, the lifting/lowering underframe further comprises a fixed casing and a movable casing, wherein the fixed casing has an accommodating space, wherein the movable casing has an receiving space, wherein the fixed casing is fixed on the affixed base, wherein the lifting/lowering mechanism is held in the accommodating space, wherein the movable casing is fixed to the lifting/lowering mechanism, wherein the lifting/lowering mechanism is held in the receiving space, wherein the movable casing is movably mounted on the fixed casing, wherein the movable casing rises and descends relative to the fixed casing following the lifting/lowering mechanism.

According to an embodiment of the present invention, the driving member is implemented as an electric push rod.

According to another aspect of the present invention, the present invention provides a load-bearing device with a lifting/lowering underframe, which comprises:
a lifting/lowering mechanism, which comprises a first lifting/lowering frame, a second lifting/lowering frame and a driving member, wherein two ends of the driving member are respectively connected with the first lifting/lowering frame and the second lifting/lowering frame, wherein the driving member is an electric push rod, wherein the driving member held between the first lifting/lowering frame and the second lifting/lowering frame allows the relative movement of the first lifting/lowering frame and the second lifting/lowering frame to be driven electrically; and
a load-bearing device, which is installed above the lifting/lowering mechanism.

According to another aspect of the present invention, the present invention provides a load-bearing device with a lifting/lowering underframe, which comprises:
a lifting/lowering mechanism and an affixed base, wherein the lifting/lowering mechanism comprises a rotating member, a maintaining member and at least one driving member, wherein two ends of the driving member are respectively connected with the rotating member and the maintaining member, so that the driving member is capable of driving the rotating member and the maintaining member to move relative to each other, wherein the lifting/lowering mechanism is rotatably mounted on the affixed base, and allows the lifting/lowering underframe to be capable of switching between a rotatable state and a locking state; and
a load-bearing device, which is installed above the lifting/lowering mechanism.

Beneficial effects of the present invention:

The lifting/lowering mechanism of the present invention is driven by the electric push rod, which can save the use space without the support of a complex complete system, and the lifting/lowering mechanism can work safely and reliably without protection; the lifting/lowering mechanism of the present invention is simple to maintain, has low noise, and can work normally in high/low temperature, and anti-corrosion/explosion-proof harsh environments.

The lifting/lowering underframe of the present invention applies the above-mentioned lifting/lowering mechanism, when the lifting/lowering underframe needs to be lifted and lowered, the driving member drives the first lifting/lowering frame to move relative to the second lifting/lowering frame, so as to drive the first casing to move relative to the second casing, thereby realizing the height adjustment of the first casing and meeting the requirements of use. The lifting/lowering underframe of the present invention integrates lifting, rotating and rotating locking functions, has simple and compact structure, stable running process and convenient operation, and is very suitable for application in fields such as tattooing beds or tattooing chairs.

### BRIEF DESCRIPTION OF THE DRAWINGS

FIG. 1 is a first perspective view of a lifting/lowering mechanism according to an embodiment of the present invention.

FIG. 2 is a perspective view of the first lifting/lowering frame of the lifting/lowering mechanism after being raised according to FIG. 1.

FIG. 3 is an exploded view of the lifting/lowering mechanism illustrated according to FIG. 1.

FIG. 4 is a second the perspective view of lifting/lowering mechanism according to an embodiment of the present invention.

FIG. 5 is a third of the perspective view of the lifting/lowering mechanism according to an embodiment of the present invention.

FIG. 6 is a perspective view of a lifting/lowering underframe according to an embodiment of the present invention.

FIG. 7 is an exploded view of the partial structure of the lifting/lowering underframe illustrated according to FIG. 5.

FIG. 8 is a perspective view of the partial structure of the lifting/lowering underframe illustrated according to FIG. 5.

FIG. 9 is a perspective view of the lifting/lowering underframe according to another preferred embodiment of the present invention.

FIG. 10 is a perspective view of a partial structure of the lifting/lowering underframe according to the above preferred embodiment of the present invention.

FIG. 11 is an exploded view of the partial structure of the lifting/lowering underframe according to the above preferred embodiment of the present invention.

FIG. 12 is a perspective view of a load-bearing device with a lifting/lowering underframe according to the present invention.

FIG. 13A is a perspective view of an application of the load-bearing device with the lifting/lowering underframe according to the above preferred embodiment of the present invention.

FIG. 13B is a perspective view of an application of the load-bearing device with the lifting/lowering underframe according to the above preferred embodiment of the present invention.

FIG. 14A is a perspective view of an application of the load-bearing device with the lifting/lowering underframe according to the above preferred embodiment of the present invention.

FIG. 14B is a perspective view of an application of the load-bearing device with the lifting/lowering underframe according to the above preferred embodiment of the present invention.

FIG. 15 is a perspective view of the lifting/lowering underframe according to another preferred embodiment of the present invention.

FIG. 16 is a perspective view of the lifting/lowering underframe according to another preferred embodiment of the present invention.

FIG. 17 is a perspective view of the lifting/lowering underframe according to another preferred embodiment of the present invention.

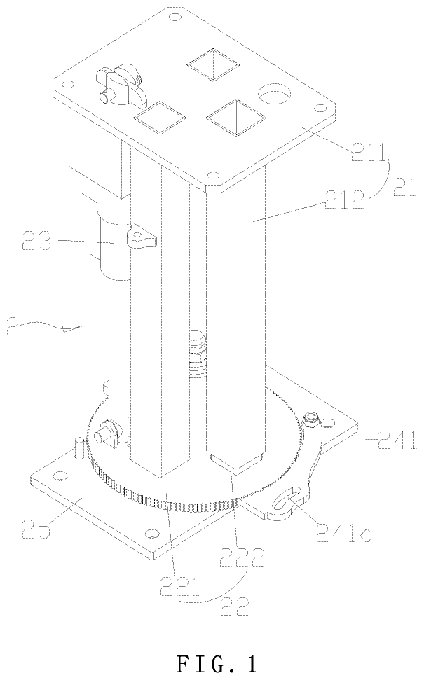

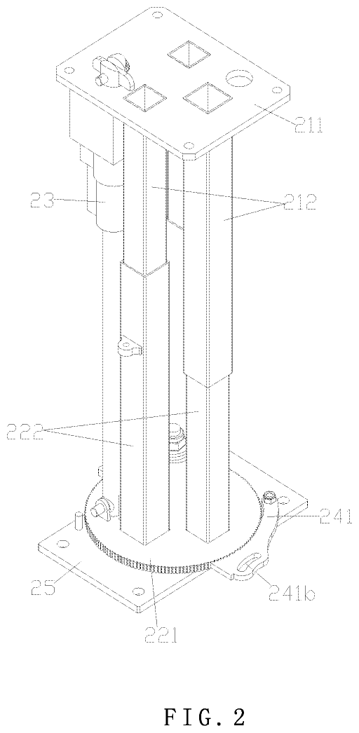

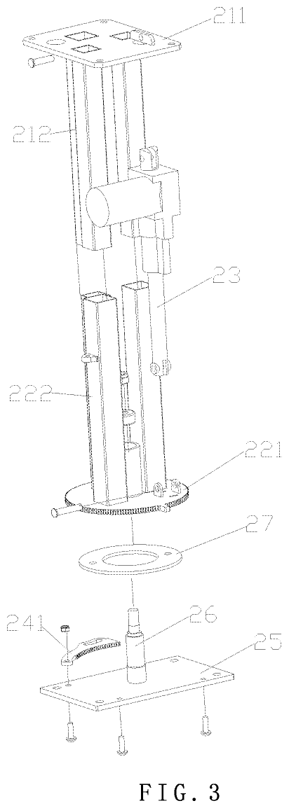

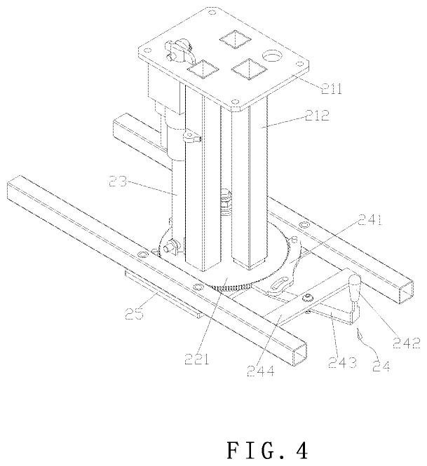

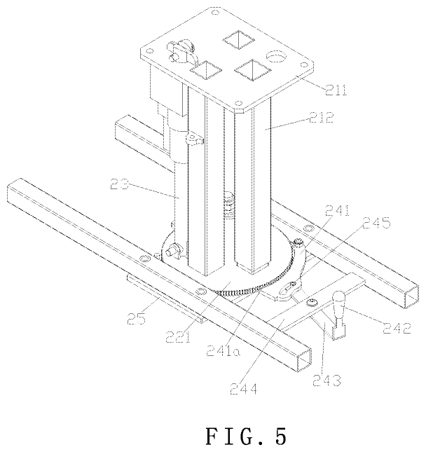

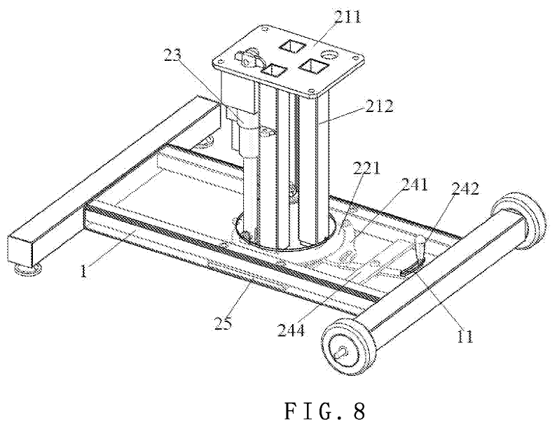

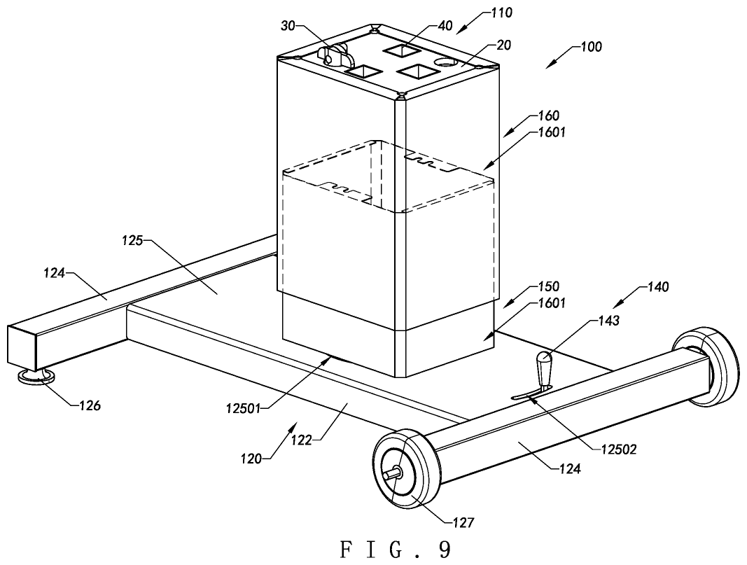

Reference signs in FIGS. 1 to 8: 1—chassis; 2—lifting/lowering mechanism; 3—first casing; 4—second casing; 11—second limiting groove; 21—first lifting/lowering frame; 22—second lifting/lowering frame; 23—driving member; 24—rotation adjusting component; 25—bottom plate; 26—rotating shaft; 27—gasket; 211—upper mounting plate; 212—first lifting/lowering pipe; 221—lower mounting plate; 222—second lifting/lowering pipe; 241—limiting block; 242—adjustment handle; 243—linkage rod; 244—affixed rod; 245—limiting column; 241a—teeth; 241b—first limiting groove.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

Those skilled in the art should understand that, in the disclosure of the present invention, terminologies of “longitudinal,” “lateral,” “upper,” “front,” “back,” “left,” “right,” “perpendicular,” “horizontal,” “top,” “bottom,” “inner,” “outer,” and etc. just indicate relations of direction or position are based on the relations of direction or position shown in the appended drawings, which is only to facilitate descriptions of the present invention and to simplify the descriptions, rather than to indicate or imply that the referred device or element must apply specific direction or to be operated or configured in specific direction. Therefore, the above-mentioned terminologies shall not be interpreted as confine to the present invention.

It is understandable that the term “a” should be understood as “at least one” or “one or more”. In other words , in one embodiment , the number of an element can be one and in other embodiment the number of the element can be greater than one. The term “a” is not construed as a limitation of quantity.

The present invention will be described in further detail below in conjunction with the accompanying drawings.

FIGS. 1 to 5 schematically illustrate a lifting/lowering mechanism according to an embodiment of the present invention.

As shown in FIGS. 1 to 5, the lifting/lowering mechanism 2 includes a first lifting/lowering frame 21, a second lifting/lowering frame 22 and a driving member 23. The first lifting/lowering frame 21 is sleeved and mounted on the second lifting/lowering frame 22. The driving member 23 of the embodiment may be an electric push rod. The driving member 23 is mounted on one of the first lifting/lowering frame 21 or the second lifting/lowering frame 22, and is used for driving the first lifting/lowering frame 21 and the second lifting/lowering frame 22 to move up and down relative to each other. The base part of the driving member 23 of the embodiment is fixedly mounted on the first lifting/lowering frame 21, and an end of the telescopic rod is mounted on the second lifting/lowering frame 22.

The lifting/lowering mechanism 2 of the present invention is driven by the electric push rod, which can save the use space without the support of a complex complete system, and the lifting/lowering mechanism can work safely and reliably without protection; the lifting/lowering mechanism 2 of the present invention is simple to maintain, has low noise, and can work normally in high/low temperature, and anti-corrosion/explosion-proof harsh environments.

The first lifting/lowering frame 21 includes an upper mounting plate 211 and a first lifting/lowering pipe 212 mounted on the upper mounting plate 211, and there is at least one first lifting/lowering pipe 212. The second lifting/lowering frame 22 includes a lower mounting plate 221 and a second lifting/lowering pipe 222 mounted on the lower mounting plate 221, and there is at least one second lifting/lowering pipe 222. The second lifting/lowering pipe 222 is sleeved and installed on the first lifting/lowering pipe 212. There are two first lifting/lowering pipes 212 in the embodiment, and they are both square tubes. There are also two second lifting/lowering pipes 222, and they are all square tubes. One of the first lifting/lowering pipes 212 is sheathed inside one of the second lifting/lowering pipes 222 , and the other first lifting/lowering pipe 212 is sheathed outside the other second lifting/lowering pipe 222. Thereby, the smooth progress of the lifting/lowering process can be ensured, and the safety is further improved. Two ends of the driving member 23 are respectively fixedly connected with the upper mounting plate 211 and the lower mounting plate 221 through fasteners such as pins.

The lifting/lowering mechanism 2 of the embodiment further includes a rotation adjusting component 24 and a bottom plate 25. The rotation adjustment assembly 24 includes a limiting block 241 arranged thereon. The lower mounting plate 221 is a gear plate. Both the limiting block 241 and the lower mounting plate 221 are rotatably mounted on the bottom plate 25, and the limiting block 241 is located an outer side of the lower mounting plate 221. The limiting block 241 is limitedly matched with the lower mounting plate 221. Consequently, the lifting/lowering mechanism 2 of the present invention not only has a height lifting/lowering function, but also has a rotation adjustment function and a rotation limit function, which has stronger applicability and more complete functions, and can meet various usage requirements.

The limiting block 241 has a plurality of teeth 241a disposed thereon and matched with the lower mounting plate 221. When locked, the teeth 241a engage with the teeth on the outer periphery of the lower mounting plate 221. Therefore, the limiting block 241 can better limit and lock the lower mounting plate 221.

The lower mounting plate 221 is rotatably mounted on the bottom plate 25 via a rotating shaft 26, a bearing, and the like. Therefore, the lower mounting plate 221 can drive the entire lifting/lowering mechanism 2 to rotate, so as to meet the usage requirements.

A gasket 27 is provided between the lower mounting plate 221 and the bottom plate 25. Hence, the gasket 27 can effectively avoid the direct contact between the lower mounting plate 221 and the bottom plate 25, thereby prolonging the service life of each. The gasket 27 of the present invention can be a nylon gasket 27, and the nylon gasket 27 is low cost and durable.

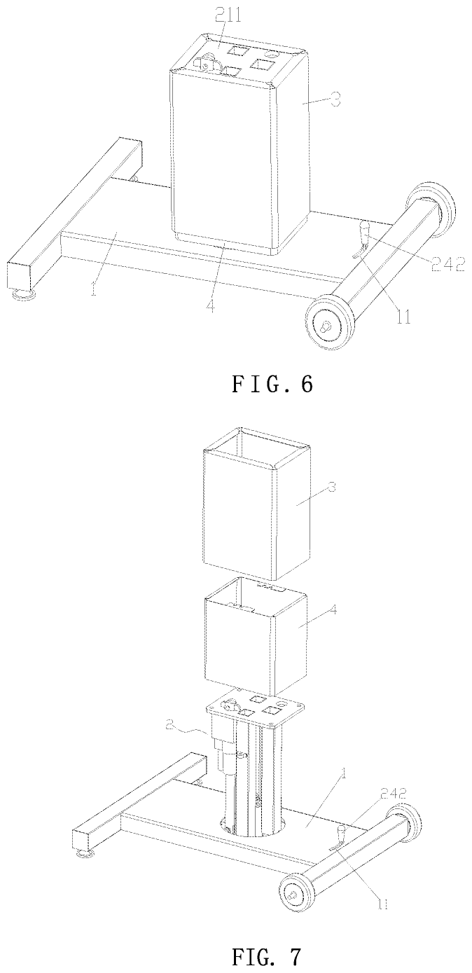

FIGS. 6-8 schematically illustrate a lifting/lowering underframe according to an embodiment of the present invention.

As shown in FIGS. 6-8, the lifting/lowering underframe includes the lifting/lowering mechanism 2 mentioned above. The lifting/lowering underframe further includes a chassis 1, a first casing 3 and a second casing 4. The second lifting/lowering frame 22 is mounted on the chassis 1 through the bottom plate 25, the first casing 3 is fixedly sleeved and mounted on the outer periphery of the first lifting/lowering frame 21, the second casing 4 is fixedly sleeved and mounted on the outer periphery of the second lifting/lowering frame 22. The first casing 3 is sleeved and mounted on the second casing 4. The first casing 3 of the embodiment is fixedly connected with the upper mounting plate 211 by fasteners such as screws, the second casing 4 is fixedly connected with the outer wall of the second lifting/lowering pipe 222 by fasteners such as screws.

The rotation adjusting component 24 of the embodiment further includes an adjustment handle 242, a linkage rod 243 and an affixed rod 244 . The affixed rod 244 is fixedly mounted on the chassis 1. One end of the linkage rod 243 is matched with the limiting block 241, and the other end is connected with the adjustment handle 242. The middle part of the linkage rod 243 is hinged with the affixed rod 244 by rivets or the like. Thus, the linkage rod 243 is driven to rotate by the adjustment handle 242, thereby driving the limiting block 241 to rotate, so that the teeth 241a on the limiting block 241 are away from or fastened to the lower mounting plate 221, so as to realize the unlocking and locking functions. The invention adopts the linkage mode to realize the limit adjustment, so that the operation is convenient.

The limiting block 241 has an arc-shaped first limiting groove 241b disposed thereon, wherein the opening of the first limiting groove 241b faces the linkage rod 243. The linkage rod 243 includes a limiting column 245 fixedly arranged at one end thereof to match with the first limiting groove 241b. Accordingly, the limiting column 245 can slide in the first limiting groove 241b, thereby pushing the limiting block 241 to move to complete the corresponding action.

The chassis 1 has an arc-shaped second limiting groove 11 disposed thereon, wherein the opening of the second limiting groove 11 faces opposite to the opening of the first limiting groove 241b. One end of the adjustment handle 242 is fixedly connected with the linkage rod 243, and the other end passes through the second limiting groove 11 and can move in the second limiting groove 11. Therefore, the second limiting groove 11 can limit the adjustment handle 242.

The lifting/lowering underframe of the present invention applies the above-mentioned lifting/lowering mechanism 2, when the lifting/lowering underframe needs to be lifted and lowered, the driving member 23 drives the first lifting/lowering frame 21 to move relative to the second lifting/lowering frame 22, so as to drive the first casing 3 to move relative to the second casing 4, thereby realizing the height adjustment of the first casing 3 and meeting the requirements of use. The lifting/lowering underframe of the present invention integrates lifting, rotating and rotating locking functions, has simple and compact structure, stable running process and convenient operation, and is very suitable for application in fields such as tattooing beds or tattooing chairs.

The foregoing are merely some of the embodiments of the present invention. For those of ordinary skill in the art, under the premise of not departing from the inventive concept of the present invention, several modifications and improvements can also be made, which all belong to the protection scope of the present invention.

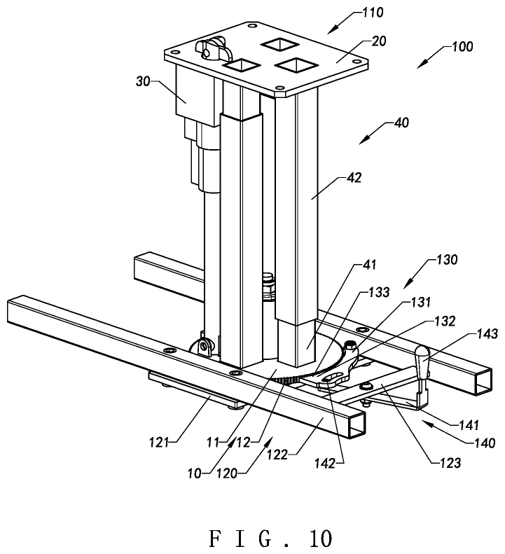

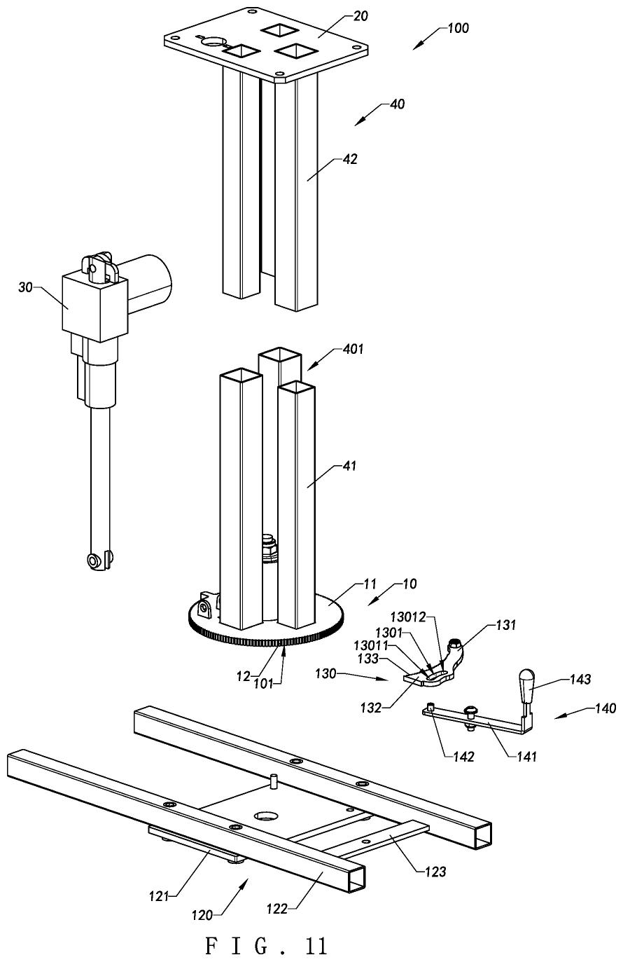

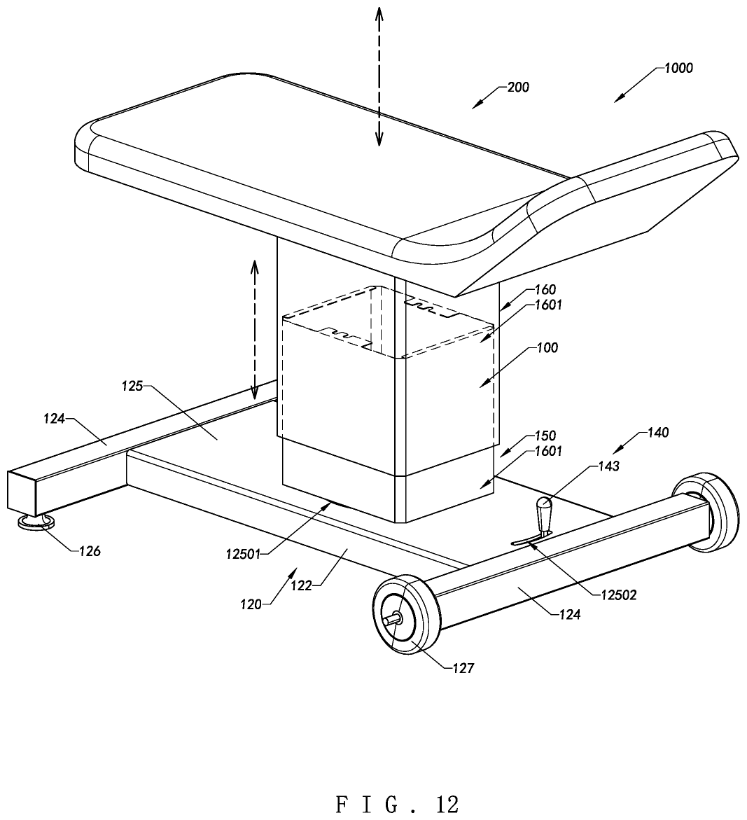

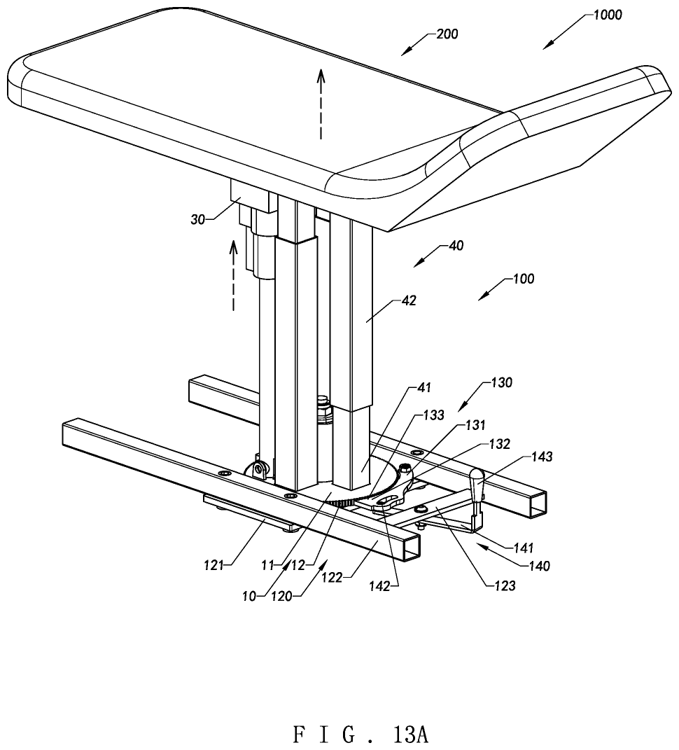

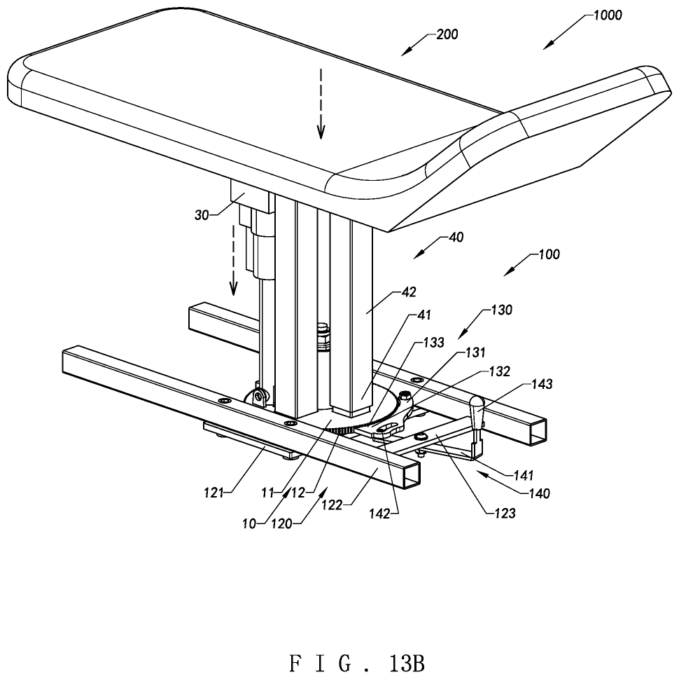

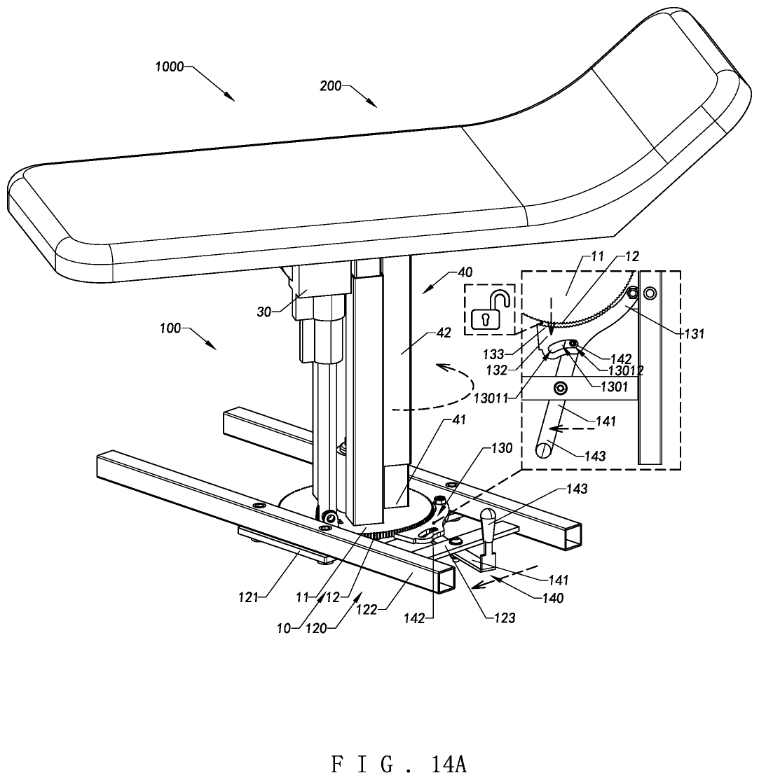

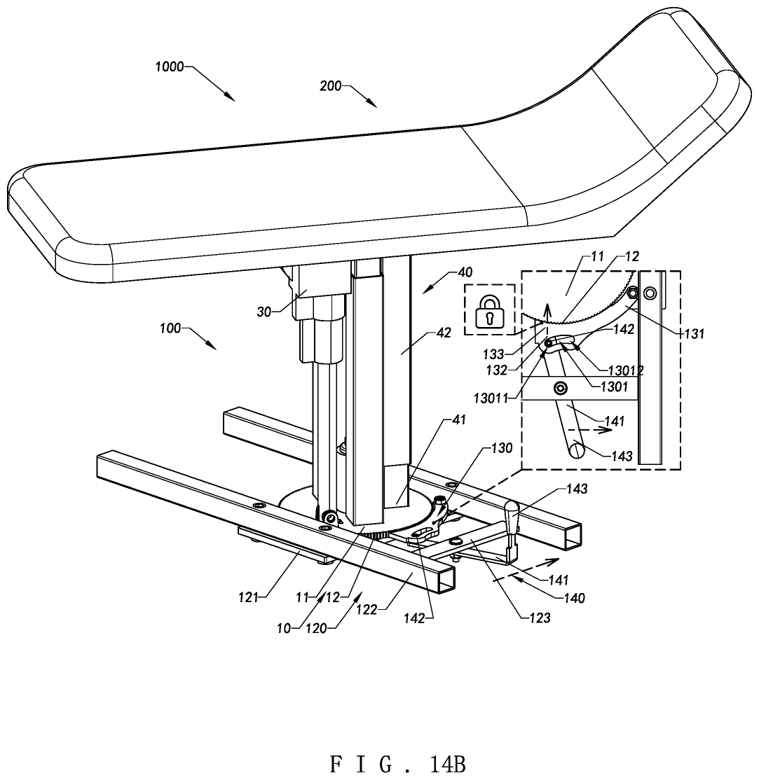

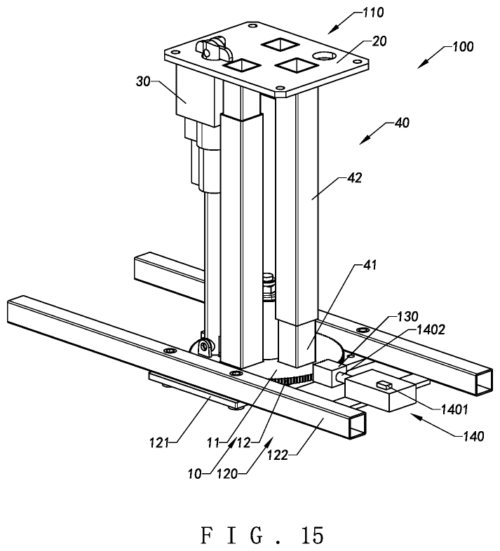

Referring to FIGS. 9 to 14B, a lifting/lowering underframe 100 according to a preferred embodiment of the present invention will be described in the following description, wherein the lifting/lowering underframe 100 is suitable for a load-bearing device 200, the load-bearing device 200 is arranged above the lifting/lowering underframe 100, and the lifting/lowering underframe 100 can stably ascend and descend to change the height of the load-bearing device 200. Moreover, the lifting/lowering underframe 100 can be switched between a rotatable state and a locking state. The lifting/lowering underframe 100 in the rotatable state can be rotated, thereby changing the orientation of the load-bearing device 200; the lifting/lowering underframe 100 in the locking state is fixed and cannot be rotated, so that the orientation of the load-bearing device 200 is fixed.

According to a specific embodiment of the present invention, the load-bearing device 200 is implemented as a tattooing bed or a tattooing chair. In the process of tattooing, the tattoo artist can change the height of the load-bearing device 200 by operating the lifting/lowering underframe 10, so that the load-bearing device 200 can always be maintained at a suitable operating height, can be adapted to tattoo artists of different heights and customers of different heights, and is also convenient for tattoo artists to operate. Furthermore, the tattoo artist can adjust the orientation of the load-bearing device 200 by rotating the lifting/lowering underframe 10, thereby changing the position of the tattoo. It is worth mentioning that the specific implementation of the load-bearing device 200 is not limited, and the load-bearing device 200 may also be implemented as a shelf, a table, or the like.

Specifically, the lifting/lowering underframe 100 includes a lifting/lowering mechanism 110 and a affixed base 120, wherein the lifting/lowering mechanism 110 is movably installed above the affixed base 120, and the load-bearing device 200 is installed on the lifting/lowering mechanism 110. The lifting/lowering mechanism 110 can move up and down relative to the affixed base 120, thereby driving the load-bearing device 200 to ascend and descend. Moreover, the lifting/lowering mechanism 110 is rotatably mounted on the affixed base 120, and the orientation of the load-bearing device 200 can be adjusted by driving the lifting/lowering mechanism 110 to rotate relative to the affixed base 120.

More specifically, the lifting/lowering mechanism 110 includes a rotating member 10, a maintaining member 20 and at least one driving member 30,
wherein two ends of the driving member 30 are respectively connected with the rotating member 10 and the maintaining member 20, the driving member 30 is held between the rotating member 10 and the maintaining member 20, and the driving member 30 can drive the rotating member 10 and the maintaining member 20 to move relative to each other, so that the height of the lifting/lowering underframe 100 is adjusted by changing the relative distance between the rotating member 10 and the maintaining member 20, thereby adjusting the height of the load-bearing device 200.

According to a specific embodiment of the present invention, the driving member 30 is implemented as an electric push rod, which has a simple structure, is safe and reliable, saves space, is easy to maintain, has low noise, and can work normally in high/low temperature and anti-corrosion/explosion-proof harsh environments. The driving member 30 electrically drives the rotating member 10 and the maintaining member 20 to move relative to each other. It should be understood by those skilled in the art that the driving member 30 of the specific implementation is only an example, cannot be a limitation on the content and scope of the lifting/lowering underframe 100 and the load-bearing device 1000 with the lifting/lowering underframe of the present invention. The drive member 30 can also be implemented as other devices for driving up and down known to those skilled in the art.

Preferably, referring to FIGS. 9 to 14B, the number of the driving member 30 is implemented as one, and the rotating member 10 and the maintaining member 20 are driven to move relative to each other by one of the driving members 30 . Optionally, referring to FIG. 17, the number of the driving member 30 is implemented as two, wherein two the driving members 30 are symmetrically distributed on both sides of the central axis of the rotating member 10 and the maintaining member 20, and the two driving members 30 can synchronously drive the rotating member 10 and the maintaining member 20 to approach each other or move away from each other, which is beneficial to the smooth relative movement of the rotating member 10 and the maintaining member 20. It should be understood by those skilled in the art that, by increasing the number of the driving members 30, the weight that the lifting/lowering underframe 100 can carry can be increased and the lifting/lowering power can be improved. Additionally, the specific implementation and specific number of the driving members 30 are only examples, and cannot be a limitation on the content and scope of the lifting/lowering underframe 100 of the present invention, and the driving members 30 may also be implemented in three or other numbers.

According to this specific embodiment of the present invention, the lifting/lowering mechanism 110 of the lifting/lowering underframe 100 further comprises at least one holding member 40, wherein the holding member 40 includes a first holder 41 and a second holder 42, wherein the first holder 41 is fixed to the rotating member 10, the second holder 42 is fixed to the maintaining member 20, wherein the first holder 41 is mounted on the second holder 42 so as to be movable up and down. In this way, it is beneficial to ensure that the lifting/lowering underframe 100 rises and falls smoothly.

Preferably, the first holder 41 has a holding space 401,
wherein the second holder 42 is installed in the holding space 401 of the first holder 41. When the driving member 30 drives the rotating member 10 and the maintaining member 20 to move relatively, the second holder 42 moves up and down along the extending direction of the holding space 401 of the first holder 41. In this way, the first holder 41 can guide and limit the movement direction of the second holder 42, so as to avoid the deviation of the second holder 42 during the movement, thereby helping to ensure that the lifting/lowering underframe 100 rises and falls stably.

Preferably, the second holder 42 has the holding space 401, wherein the first holder 41 is installed in the holding space 401 of the second holder 42. When the driving member 30 drives the rotating member 10 and the maintaining member 20 to move relatively, the first holder 41 moves up and down along the extending direction of the holding space 401 of the second holder 42. In this way, the second holder 42 can guide and limit the movement direction of the first holder 41, so as to avoid the deflection of the first holder 41 during the movement.

Preferably, the holding members 40 are implemented as three, wherein the three holding members 40 are held between the rotating member 10 and the maintaining member 20 at a distance from each other, and the three holding members 40 form a stable triangular structure, so as to further improve the stability of the lifting/lowering underframe 100. It should be understood by those skilled in the art that the specific number of the holding members 40 is not limited, wherein the holding members 40 can also be implemented as one, two, four or other numbers. The specific number of the holding members 40 is only an example, and cannot be a limitation on the content and scope of the lifting/lowering underframe 100 of the present invention.

Preferably, the first holder 41 and the second holder 42 of the holding member 40 are implemented as square tubes, that is, the cross sections of the first holder 41 and the second holder 42 are square rings. Optionally, the first holder 41 and the second holder 42 of the holding member 40 are implemented as circular tubes, that is, the cross sections of the first holder 41 and the second holder 42 are circular rings.

It is worth mentioning that the specific shapes of the first holder 41 and the second holder 42 are not limited, wherein the cross sections of the first holder 41 and the second holder 42 can also be implemented as triangular, trapezoidal, elliptical, or the like. The first holder 41 and the second holder 42 can be implemented as hollow or solid. It should be understood by those skilled in the art that the specific structures of the first holder 41 and the second holder 42 are only examples, and cannot be a limitation on the content and scope of the lifting/lowering underframe 100 of the present invention.

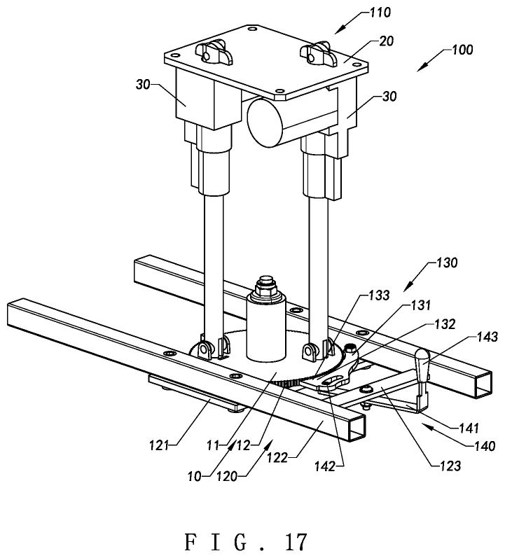

Preferably, referring to FIG. 16, the holding member 40 further comprises a linear bearing 43, wherein the linear bearing 43 is mounted on the first holder 41 and/or the second holder 42, and the linear bearing 43 cooperates with the first holder 41 and the second holder 42, so that the first holder 41 and the second holder 42 maintain a smooth linear motion, which is further beneficial to improve the stability of the lifting/lowering underframe 100.

Optionally, the first holder 41 of the holding member 40 is implemented as the linear bearing 43, and the second holder 42 is implemented as a circular tube, wherein the second holder 42 can move up and down relative to the linear bearing 43. Optionally, the second holder 42 of the holding member 40 is implemented as the linear bearing 43, and the first holder 41 is implemented as a circular tube, wherein the first holder 41 may move up and down relative to the linear bearing 43.

According to a specific embodiment of the present invention, the maintaining member 20 of the lifting/lowering mechanism 110 is located above the rotating member 10, and the rotating member 10 is rotatably mounted on the affixed base 120. By driving the rotating member 10 to rotate relative to the affixed base 120, the lifting/lowering mechanism 110 can be rotated relative to the affixed base 120, thereby changing the orientation of the load-bearing device 200 installed on the lifting/lowering mechanism 110.

The lifting/lowering underframe 100 according to the specific embodiment of the present invention, the rotating member 10 includes an assembly portion 11 and a plurality of mutually spaced limiting teeth 12 extending outward from the edge of the assembly portion 11, and a limiting tooth slot 101 is formed between two adjacent limiting teeth 12 and the assembly portion 11.

The lifting/lowering underframe 100 further includes a limiting member 130 and an operating member 140, wherein the operating member 140 can drive at least a part of the limiting member 130 to enter the limiting tooth slot 101 or leave the limiting tooth slot 101. Moreover, after at least a part of the limiting member 130 enters the limiting tooth slot 101, the limiting member 130 restricts the rotation of the lifting/lowering mechanism 110 relative to the affixed base 120, the lifting/lowering underframe 100 is in the locking state, and the orientation of the load-bearing device 200 is fixed. In addition, when the limiting member 130 is away from the limiting tooth slot 101, the limiting member 130 is separated from the limiting tooth slot 101, the lifting/lowering underframe 100 is switched to the rotatable state, and the lifting/lowering mechanism 110 of the lifting/lowering underframe 100 is allowed to rotate relative to the affixed base 120.

Referring to FIGS. 9 to 14B, according to a specific embodiment of the present invention, the operating member 140 allows manual operation to manually drive the limiting member 130 close to or away from the limiting teeth 12. In this way, the user makes the lifting/lowering underframe 100 switch between the locking state and the rotatable state through manual control. Specifically, the limiting member 130 includes a rotating portion 131, a limiting arm 132 bent outwardly extending from the rotating portion 131, and at least one limiting protrusion 133 extending outwardly from the inner wall of the limiting arm 132. The limiting member 130 has a driving space 1301, wherein the driving space 1301 is formed in the limiting arm 132, the rotating portion 131 of the limiting member 130 is rotatably mounted on the affixed base 120, and the limiting protrusion 133 is movably retained on the limiting tooth slot 101 of the rotating member 10.

The operating member 140 includes a connecting arm 141, a limiting column 142 and an operating portion 143, wherein the limiting column 142 and the connecting arm 141 respectively extend outward from two ends of the connecting arm 141. The connecting arm 141 is rotatably mounted on the affixed base 120 in a manner that the limiting column 142 is movably held in the driving space 1301 of the limiting member 130. By operating the operating portion 143, the connecting arm 141 can be driven to rotate relative to the affixed base 120, and the limiting column 142 can be driven to move within the driving space 1301 of the limiting member 130, so that the limiting column 142 drives the limiting member 130 to be close to the rotating member 10 or away from the rotating member 10.

The driving space 1301 has a driving proximal end 13011 and a driving distal end 13012 relative to the driving proximal end 13011, wherein the vertical distance between the driving proximal end 13011 and the limiting protrusion 133 is greater than the vertical distance between the driving distal end 13012 and the limiting protrusion 133. When the limiting column 142 of the operating member 140 is located at the driving proximal end 13011 of the driving space 1301, the limiting protrusion 133 of the limiting member 130 enters the limiting tooth slot 101 of the rotating member 10, so that the lifting/lowering mechanism 110 cannot rotate relative to the affixed base 120. When the limiting column 142 of the operating member 140 moves from the driving proximal end 13011 of the driving space 1301 to the driving distal end 13012, the rotating portion 131 of the limiting member 130 is driven by the limiting column 142 to rotate counterclockwise relative to the affixed base 120, and the limiting protrusion 133 leaves the limiting tooth slot 101, so that the lifting/lowering mechanism 110 is allowed to rotate relative to the affixed base 120. After the lifting/lowering mechanism 110 is rotated to an appropriate position, the operating portion 143 of the operating member 140 is toggled, and the connecting arm 141 rotates relative to the affixed base 120, and then the limiting column 142 moves from the driving distal end 13012 of the driving space 1301 to the driving proximal end 13011, so that the limiting protrusion 133 is driven close to the rotating member 10 and enters the limiting tooth slot 101 of the rotating member 10.

Preferably, the driving space 1301 of the limiting member 130 is an arc-shaped groove, and the limiting column 142 moves along the arc-shaped surface, which facilitates the user to adjust the state of the lifting/lowering underframe 100 easily and effortlessly. Optionally, the driving space 1301 is a square groove. It should be understood by those skilled in the art that the specific implementation of the driving space 1301 of the limiting member 130 is only an example, and cannot be a limitation on the content and scope of the lifting/lowering underframe 100 of the present invention.

According to this specific embodiment of the present invention, the affixed base 120 of the lifting/lowering underframe 100 includes a fixing element 121, two lateral support elements 122, an assembly element 123, two longitudinal support elements 124 and a cover element 125 arranged thereon, wherein the over element 125 includes an assembly through hole 12501 and an operation space 12502 arranged thereon. Two ends of the fixing element 121 are affixed to the two lateral support elements 122 respectively, and the rotating member 10 is rotatably mounted on the fixing element 121. For example, the rotating member 10 is rotatably mounted on the fixed plate 121 through a rotating shaft, a bearing, and the like.

The rotating portion 131 of the limiting member 130 is rotatably mounted on the fixing element 121 and is held on one side of the rotating member 10. Two ends of the assembly element 123 are respectively connected with the two lateral support elements 122, and the assembly element 123 is located on one side of the fixing element 121. The connecting arm 141 of the operating member 140 is rotatably mounted on the assembly element 123. The assembly through hole 12501 and the operation space 12502 of the over element 125 are respectively installed above the lateral support elements 122 in a manner corresponding to the holding member 40 and the operating portion 143 of the operating member 140. The operating portion 143 of the operating member 140 can be driven to move within the operation space 12502. The longitudinal support elements 124 are respectively disposed on two ends of the lateral support elements 122 and form an “H”-shaped structure, which improves the stability of the lifting/lowering underframe 100.

Preferably, the affixed base 120 further includes two protective elements 126 and two wheels 127 respectively disposed thereon, wherein two of the protective elements 126 are placed under one of the longitudinal support elements 124 at intervals, and two of the wheels 127 are installed at two ends of the other of the longitudinal support elements 124 at intervals. In this way, the wear of the lifting/lowering underframe 100 on the ground can be reduced by the use of the protective elements 126, and the lifting/lowering underframe 100 can be easily pushed by the wheels 127.

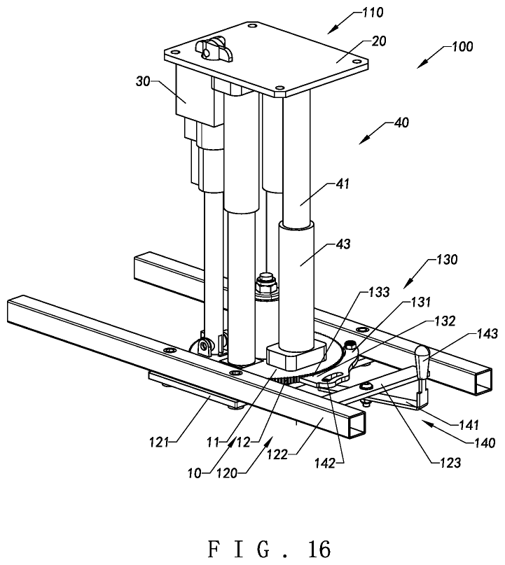

Referring to FIG. 15, according to a specific embodiment of the present invention, the operating member 140 can electrically drive the limiting member 130 into the limiting tooth slot 101 of the rotating member 10 or leave the limiting tooth slot 101. Specifically, the operating member 140 includes a control member 1401 and an electric telescopic member 1402, wherein the control member 1401 is communicatively connected with the electric telescopic member 1402, wherein the limiting member 130 is arranged at the end of the electric telescopic member 1402, wherein the control member 140 can control the telescopic length of the electric telescopic member 1402, so that at least a part of the limiting member 130 can enter the limiting tooth slot 101 or leave the limiting tooth slot 101. In this way, the lifting/lowering underframe 1000 can be switched between the locking state and the rotatable state.

Specifically, when the lifting/lowering underframe 100 is in the locked state, the electric telescopic member 1402 is in an extended state, and the limiting protrusion 133 of the limiting member 130 disposed at the end of the electric telescopic member 1402 enters into the limiting tooth slot 101 of the rotating member 10, so that the lifting/lowering mechanism 110 cannot rotate relative to the affixed base 120. When the control member 1401 is operated to switch the electric telescopic member 1402 from the extended state to the retracted state, the limiting member 130 moves with the electric telescopic member 1402, and the limiting protrusion 133 of the limiting member 130 leaves the limiting tooth slot 101 of the rotating member 10. Preferably, the control member 1401 is implemented as a button, and the length of the electric telescopic member 1402 can be changed by pressing the button. Preferably, the control member 1401 is implemented as a knob, and the length of the electric telescopic member 1402 can be adjusted by twisting the knob.

Optionally, the maintaining member 20 of the lifting/lowering mechanism 110 is located below the rotating member 10, the maintaining member 20 is rotatably mounted on the affixed base 120, and the limiting member 130 and the operating member 140 can cooperate with each other so that the lifting/lowering underframe 100 can be switched between the rotatable state and the locking state.

The lift lifting/lowering underframe 100 further includes a fixed casing 150 and a movable casing 160, wherein the fixed casing 150 has an accommodating space 1501 arranged thereon, and the movable casing 160 has a receiving space 1601 arranged thereon. The fixed casing 150 is affixed to the affixed base 120, and the lifting/lowering mechanism 110 is held in the accommodating space 1501. The movable casing 160 is affixed to the lifting/lowering mechanism 110, and the lifting/lowering mechanism 110 is held in the receiving space 1601. The movable casing 160 is movably mounted on the fixed casing 150, and the movable casing 160 follows the lifting/lowering mechanism 110 to ascend and descend relative to the fixed casing 150, wherein the lifting/lowering mechanism 110 is always hidden inside the fixed casing 150 and the movable casing 160, which is beneficial to protect the lifting/lowering mechanism 110 and also prevent the user from being pinched by the driving member 130 and the maintaining member 140 that move up and down.

According to a specific embodiment of the present invention, the fixed casing 150 is held in the receiving space 1601 of the movable casing 160, that is, the movable casing 160 is movably sleeved and mounted on the fixed casing 150 up and down. Optionally, the movable casing 160 is held in the accommodating space 1501 of the fixed casing 150, that is, the fixed casing 150 is sheathed on the movable casing 160.

Those skilled in the art can understand that the above embodiments are only examples, wherein the features of different embodiments can be combined with each other to obtain embodiments that are easily conceived according to the disclosure of the present invention but are not clearly indicated in the accompanying drawings.

One skilled in the art will understand that the embodiment of the present invention as shown in the drawings and described above is exemplary only and not intended to be limiting. It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

1. A lifting/lowering underframe, comprising:
a lifting/lowering mechanism, which comprises a first lifting/lowering frame, a second lifting/lowering frame and a driving member, wherein two ends of said driving member are respectively connected with said first lifting/lowering frame and said second lifting/lowering frame, wherein said driving member is an electric push rod, wherein said driving member held between said first lifting/lowering frame and said second lifting/lowering frame allows the relative movement of said first lifting/lowering frame and said second lifting/lowering frame to be driven electrically.

2. The lifting/lowering underframe, as recited in claim 1, wherein said first lifting/lowering frame comprises an upper mounting plate and at least one first lifting/lowering pipe extending downward from said upper mounting plate, wherein said second lifting/lowering frame comprises a lower mounting plate and at least one second lifting/lowering pipe mounted on said lower mounting plate, wherein said first lifting/lowering frame is held above said lower mounting plate in a manner that said first lifting/lowering pipe is movably mounted on said second lifting/lowering pipe, wherein said two ends of said driving member are respectively connected with said upper mounting plate and said lower mounting plate.

3. The lifting/lowering underframe, as recited in claim 2, wherein said lifting/lowering underframe further comprises a limiting block and a bottom plate, wherein said lower mounting plate and said limiting block are rotatably mounted on said bottom plate, and said limiting block is located an outer side of said lower mounting plate, so that said limiting block is allowed to limit the rotation of said lower mounting plate relative to said bottom plate.

4. The lifting/lowering underframe, as recited in claim 3, wherein said limiting block of said lifting/lowering underframe has a plurality of mutually spaced tooth slots formed inwardly recessed on said surface thereof, wherein said limiting block has at least one tooth matched with said tooth slots and arranged thereon.

5. The lifting/lowering underframe, as recited in claim 3, wherein said lifting/lowering underframe comprises a chassis for fixing said bottom plate, a first casing sleeved and mounted on said first lifting/lowering frame, and a second casing sleeved and mounted on said second lifting/lowering frame, wherein said first casing is movably mounted to said second casing.

6. The lifting/lowering underframe, as recited in claim 5, wherein said lifting/lowering underframe further comprises a affixed rod fixed to said chassis, a linkage rod movably arranged on said affixed rod, and an adjustment handle arranged at one end of said linkage rod, wherein the other end of said linkage rod is connected with said limiting block, so that said linkage rod is capable of driving said limiting block to move relative to said lower mounting plate.

7. The lifting/lowering underframe, as recited in claim 6, wherein said lifting/lowering underframe further comprises a limiting column arranged at said end of said linkage rod, wherein said limiting block has a first limiting groove arranged thereon, wherein said limiting column is movably held in said first limiting groove of said limiting block.

8. The lifting/lowering underframe, as recited in claim 7, wherein said chassis has a second limiting groove arranged thereon, wherein said adjustment handle is movably held in said second limiting groove.

9. A lifting/lowering underframe, comprising:
a lifting/lowering mechanism, which comprises a rotating member, a maintaining member and at least one driving member, wherein two ends of said driving member are respectively connected with said rotating member and said maintaining member, so that said driving member is capable of driving said rotating member and said maintaining member to move relative to each other; and
an affixed base, wherein said lifting/lowering mechanism is rotatably mounted on said affixed base, and allows said lifting/lowering underframe to be capable of switching between a rotatable state and a locking state.

10. The lifting/lowering underframe, as recited in claim 9, wherein said lifting/lowering mechanism further comprises at least one holding member, wherein said holding member comprises a first holder affixed to said rotating member, and a second holder affixed to said maintaining member, wherein said second holder is movably mounted to said first holder.

11. The lifting/lowering underframe, as recited in claim 10, wherein said lifting/lowering underframe further comprises a linear bearing mounted on said first holder or said second holder.

12. The lifting/lowering underframe, as recited in claim 9, wherein said driving member is implemented as two, wherein said two driving members are symmetrically arranged on said rotating member and said maintaining member.

13. The lifting/lowering underframe, as recited in claim 9, wherein said lifting/lowering underframe further comprises a limiting member, wherein said rotating member comprises an assembly portion, a plurality of mutually spaced limiting teeth extending outward from an edge of said assembly portion, and a limiting tooth slot is formed between said two adjacent limiting teeth and said assembly portion, wherein said limiting member is movably held on an outer side of said rotating member and at least a part of said limiting member is allowed to enter said limiting tooth slot.

14. The lifting/lowering underframe, as recited in claim 13, wherein said limiting member comprises a rotating portion, a limiting arm bent outwardly extending from said rotating portion, and at least one limiting protrusion extending outwardly from an inner wall of said limiting arm, so that after said limiting member is driven close to said rotating member, said limiting protrusion of said limiting arm is allowed to enter said limiting tooth slot of said rotating member to limit the rotation of said rotating member.

15. The lifting/lowering underframe, as recited in claim 14, wherein said lifting/lowering underframe further comprises an operating member, wherein said limiting member is drivably connected with said operating member, so that said operating member is capable of driving said limiting member to approach said rotating member and move away from said rotating member.

16. The lifting/lowering underframe, as recited in claim 15, wherein said limiting member has a driving space formed in said limiting arm, wherein said operating member comprises a connecting arm, a limiting column and an operating portion, wherein said limiting column and said connecting arm respectively extend outward from two ends of said connecting arm, wherein said connecting arm is rotatably mounted on said affixed base in a manner that said limiting column is movably held in said driving space of said limiting member, wherein said connecting arm rotates relative to said affixed base, which is capable of driving said limiting column to move in said driving space of said limiting member, and drive said limiting member to be close to said rotating member or away from said rotating member.

17. The lifting/lowering underframe, as recited in claim 16, wherein said driving space has a driving proximal end and a driving distal end relative to said driving proximal end, wherein the vertical distance between said driving proximal end and said limiting protrusion is greater than the vertical distance between said driving distal end and said limiting protrusion.

18. The lifting/lowering underframe, as recited in claim 17, wherein said driving space is an arc-shaped groove.

19. The lifting/lowering underframe, as recited in claim 15, wherein said operating member comprises a control member and an electric telescopic member, wherein said control member is communicatively connected with said electric telescopic member, wherein said limiting member is arranged at an end of said electric telescopic member, wherein said operating member electrically drives said limiting member to move relative to said rotating member.

20. The lifting/lowering underframe, as recited in claim 9, wherein said lifting/lowering underframe further comprises a fixed casing and a movable casing, wherein said fixed casing has an accommodating space, wherein said movable casing has an receiving space, wherein said fixed casing is fixed on said affixed base, wherein said lifting/lowering mechanism is held in said accommodating space, wherein said movable casing is fixed to said lifting/lowering mechanism, wherein said lifting/lowering mechanism is held in said receiving space, wherein said movable casing is movably mounted on said fixed casing, wherein said movable casing rises and descends relative to said fixed casing following said lifting/lowering mechanism.

21. The lifting/lowering underframe, as recited in claim 9, wherein said driving member is implemented as an electric push rod.

22. A load-bearing device with a lifting/lowering underframe, comprising:
a lifting/lowering mechanism, which comprises a first lifting/lowering frame, a second lifting/lowering frame and a driving member, wherein two ends of said driving member are respectively connected with said first lifting/lowering frame and said second lifting/lowering frame, wherein said driving member is an electric push rod, wherein said driving member held between said first lifting/lowering frame and said second lifting/lowering frame allows the relative movement of said first lifting/lowering frame and said second lifting/lowering frame to be driven electrically; and
a load-bearing device, which is installed above said lifting/lowering mechanism.

23. The load-bearing device with a lifting/lowering underframe, as recited in claim 22, wherein said first lifting/lowering frame comprises an upper mounting plate and at least one first lifting/lowering pipe extending downward from said upper mounting plate, wherein said second lifting/lowering frame comprises a lower mounting plate and at least one second lifting/lowering pipe mounted on said lower mounting plate, wherein said first lifting/lowering frame is held above said lower mounting plate in a manner that said first lifting/lowering pipe is movably mounted on said second lifting/lowering pipe, wherein said two ends of said driving member are respectively connected with said upper mounting plate and said lower mounting plate.

24. The load-bearing device with a lifting/lowering underframe, as recited in claim 23, wherein said lifting/lowering underframe further comprises a limiting block and a bottom plate, wherein said lower mounting plate and said limiting block are rotatably mounted on said bottom plate, and said limiting block is located an outer side of said lower mounting plate, so that said limiting block is allowed to limit the rotation of said lower mounting plate relative to said bottom plate.

25. The load-bearing device with a lifting/lowering underframe, as recited in claim 24, wherein said limiting block of said lifting/lowering underframe has a plurality of mutually spaced tooth slots formed inwardly recessed on said surface thereof, wherein said limiting block has at least one tooth matched with said tooth slots and arranged thereon.

26. The load-bearing device with a lifting/lowering underframe, as recited in claim 24, wherein said lifting/lowering underframe comprises a chassis, a first casing and a second casing, wherein said bottom plate is affixed to said chassis, said first casing is sleeved and mounted on said first lifting/lowering frame, and said second casing is sleeved and mounted on said second lifting/lowering frame, wherein said first casing is movably mounted to said second casing.

27. The load-bearing device with a lifting/lowering underframe, as recited in claim 26, wherein said lifting/lowering underframe further comprises an adjustment handle, a linkage rod and an affixed rod, wherein said adjustment handle is arranged at one end of said linkage rod, said linkage rod is movably arranged on said affixed rod, said affixed rod is affixed to the chassis, the other end of said linkage rod is connected with the limiting block, so that said linkage rod is capable of driving said limiting block to move relative to said lower mounting plate.

28. The load-bearing device with a lifting/lowering underframe, as recited in claim 27, wherein said lifting/lowering underframe further comprises a limiting column arranged at said end of said linkage rod, wherein said limiting block has a first limiting groove arranged thereon, wherein said limiting column is movably held in said first limiting groove of said limiting block.

29. The load-bearing device with a lifting/lowering underframe, as recited in claim 28, wherein said chassis has a second limiting groove arranged thereon, wherein said adjustment handle is movably held in said second limiting groove.

30. A load-bearing device with a lifting/lowering underframe, comprising:
a lifting/lowering mechanism and an affixed base, wherein said lifting/lowering mechanism comprises a rotating member, a maintaining member and at least one driving member, wherein two ends of said driving member are respectively connected with said rotating member and said maintaining member, so that said driving member is capable of driving said rotating member and said maintaining member to move relative to each other, wherein said lifting/lowering mechanism is rotatably mounted on said affixed base, and allows said lifting/lowering underframe to be capable of switching between a rotatable state and a locking state; and
a load-bearing device, which is installed above said lifting/lowering mechanism.

31. The load-bearing device with the lifting/lowering underframe, as recited in claim 30, wherein said lifting/lowering mechanism further comprises at least one holding member, wherein said holding member comprises a first holder and a second holder, wherein said first holder is affixed to said rotating member, wherein said second holder is affixed to said maintaining member, wherein said second holder is movably mounted to said first holder.

32. The load-bearing device with the lifting/lowering underframe, as recited in claim 31, wherein said lifting/lowering underframe further comprises a linear bearing, wherein said linear bearing is mounted on said first holder or said second holder.

33. The load-bearing device with the lifting/lowering underframe, as recited in claim 30, wherein said driving member is implemented as two, wherein said two driving members are symmetrically arranged on said rotating member and said maintaining member.

34. The load-bearing device with the lifting/lowering underframe, as recited in claim 30, wherein said lifting/lowering underframe further comprises a limiting member, wherein said rotating member comprises an assembly portion, a plurality of mutually spaced limiting teeth extending outward from an edge of said assembly portion, and a limiting tooth slot is formed between said two adjacent limiting teeth and said assembly portion, wherein said limiting member is movably held on an outer side of said rotating member, and at least a part of said limiting member is allowed to enter said limiting tooth slot.

35. The load-bearing device with the lifting/lowering underframe, as recited in claim 34, wherein said limiting member comprises a rotating portion, a limiting arm bent outwardly extending from said rotating portion, and at least one limiting protrusion extending outwardly from an inner wall of said limiting arm, so that after said limiting member is driven close to said rotating member, said limiting protrusion of said limiting arm is allowed to enter said limiting tooth slot of said rotating member to limit the rotation of said rotating member.

36. The load-bearing device with the lifting/lowering underframe, as recited in claim 35, wherein said lifting/lowering underframe further comprises an operating member, wherein said limiting member is drivably connected with said operating member, so that said operating member is capable of driving said limiting member to approach said rotating member and move away from said rotating member.

37. The load-bearing device with the lifting/lowering underframe, as recited in claim 36, wherein said limiting member has a driving space, wherein said driving space is formed in said limiting arm, wherein said operating member comprises a connecting arm, a limiting column and an operating portion, wherein said limiting column and said connecting arm respectively extend outward from two ends of said connecting arm, wherein said connecting arm is rotatably mounted on said affixed base in a manner that said limiting column is movably held in said driving space of said limiting member, wherein said connecting arm rotates relative to said affixed base, which is capable of driving said limiting column to move in said driving space of said limiting member, and drive said limiting member to be close to said rotating member or away from said rotating member.

38. The load-bearing device with the lifting/lowering underframe, as recited in claim 37, wherein said driving space has a driving proximal end and a driving distal end relative to said driving proximal end, wherein the vertical distance between said driving proximal end and said limiting protrusion is greater than the vertical distance between said driving distal end and said limiting protrusion.

39. The load-bearing device with the lifting/lowering underframe, as recited in claim 38, wherein said driving space is an arc-shaped groove.

40. The load-bearing device with the lifting/lowering underframe, as recited in claim 36, wherein said operating member comprises a control member and an electric telescopic member, wherein said control member is communicatively connected with said electric telescopic member, wherein said limiting member is arranged at an end of said electric telescopic member, wherein said operating member electrically drives said limiting member to move relative to said rotating member.

41. The load-bearing device with the lifting/lowering underframe, as recited in claim 30, wherein said lifting/lowering underframe further comprises a fixed casing and a movable casing, wherein said fixed casing has an accommodating space, wherein said movable casing has an receiving space, wherein said fixed casing is fixed on said affixed base, wherein said lifting/lowering mechanism is held in said accommodating space, wherein said movable casing is fixed to said lifting/lowering mechanism, wherein said lifting/lowering mechanism is held in said receiving space, wherein said movable casing is movably mounted on said fixed casing, wherein said movable casing rises and descends relative to said fixed casing following said lifting/lowering mechanism.

42. The load-bearing device with the lifting/lowering underframe, as recited in claim 30, wherein said driving member is implemented as an electric push rod.

43. The lifting/lowering underframe, as recited in any of claim 10, wherein said driving member is implemented as an electric push rod.

44. The lifting/lowering underframe, as recited in any of claim 11, wherein said driving member is implemented as an electric push rod.

45. The lifting/lowering underframe, as recited in any of claim 12, wherein said driving member is implemented as an electric push rod.

46. The lifting/lowering underframe, as recited in any of claim 13, wherein said driving member is implemented as an electric push rod.

47. The lifting/lowering underframe, as recited in any of claim 14, wherein said driving member is implemented as an electric push rod.

48. The lifting/lowering underframe, as recited in any of claim 15, wherein said driving member is implemented as an electric push rod.

49. The lifting/lowering underframe, as recited in any of claim 16, wherein said driving member is implemented as an electric push rod.

50. The lifting/lowering underframe, as recited in any of claim 17, wherein said driving member is implemented as an electric push rod.

51. The lifting/lowering underframe, as recited in any of claim 18, wherein said driving member is implemented as an electric push rod.

52. The lifting/lowering underframe, as recited in any of claim 19, wherein said driving member is implemented as an electric push rod.

53. The lifting/lowering underframe, as recited in any of claim 20, wherein said driving member is implemented as an electric push rod.

54. The load-bearing device with the lifting/lowering underframe, as recited in claim 31, wherein said driving member is implemented as an electric push rod.

55. The load-bearing device with the lifting/lowering underframe, as recited in claim 32, wherein said driving member is implemented as an electric push rod.

56. The load-bearing device with the lifting/lowering underframe, as recited in claim 33, wherein said driving member is implemented as an electric push rod.

57. The load-bearing device with the lifting/lowering underframe, as recited in claim 34, wherein said driving member is implemented as an electric push rod.

58. The load-bearing device with the lifting/lowering underframe, as recited in claim 35, wherein said driving member is implemented as an electric push rod.

59. The load-bearing device with the lifting/lowering underframe, as recited in claim 36, wherein said driving member is implemented as an electric push rod.

60. The load-bearing device with the lifting/lowering underframe, as recited in claim 37, wherein said driving member is implemented as an electric push rod.

61. The load-bearing device with the lifting/lowering underframe, as recited in claim 38, wherein said driving member is implemented as an electric push rod.

62. The load-bearing device with the lifting/lowering underframe, as recited in claim 39, wherein said driving member is implemented as an electric push rod.

63. The load-bearing device with the lifting/lowering underframe, as recited in claim 40, wherein said driving member is implemented as an electric push rod.

64. The load-bearing device with the lifting/lowering underframe, as recited in claim 41, wherein said driving member is implemented as an electric push rod.
